(19) United States
(12) Patent Application Publication
(10) Pub. No.: US 2025/0213262 A1
(43) Pub. Date: Jul. 3, 2025

(54) TONGUE CLEANER

(71) Applicant: Richard STELZER, Bischofswiesen (DE)

(72) Inventor: Richard STELZER, Bischofswiesen (DE)

(21) Appl. No.: 18/835,924

(22) PCT Filed: Feb. 3, 2023
(86) PCT No.: PCT/DE2023/100090

§ 371 Date: Aug. 5, 2024

(30) Foreign Application Priority Data
Feb. 5, 2022 (DE) 10 2022 102 726.0

(51) Int. Cl. A61B 17/24; A47L 9/06; A47L 9/28; A61B 17/00

(52) U.S. Cl. CPC A61B 17/244; A47L 9/0626; A47L 9/0693; A47L 9/2857; A61B 2017/00561; A61B 2017/00862

(57) ABSTRACT

The invention relates to a tongue cleaner for cleaning the tongue of a patient, comprising a main part and a handle section. The main part is paired with at least one cleaning region for cleaning and suctioning deposits on the tongue, wherein a first cleaning region has at least one elastic cleaning lip, which is being paired with at least one suction opening such that the suction opening is aligned at least parallel to the movement plane of the cleaning movement.

### CROSS-REFERENCE TO RELATED APPLICATIONS

This application is a US national stage under 35 USC 371 of PCT/DE2023/100090 filed on Feb. 3, 2023 which claims priority to German application no. DE 10 2022 102 726.0 filed on Feb. 5, 2022. The entire content of these applications is incorporated by reference herein.

### FIELD OF THE INVENTION

The present invention relates to a tongue cleaner for cleaning a tongue of a patient or a person, having at least one main part and a handle section, wherein the main part is paired with at least one cleaning region for cleaning and suctioning deposits on the tongue.

### BACKGROUND OF THE INVENTION

For example, from the publication DE 20 2016 001 011 U1 a tongue cleaner head with a disc-shaped body for cleaning the tongue of a patient is known. The disc-shaped body has a disc-shaped front surface on the usage side. The front surface has a profile formed to scrape off contaminations adhering to the surface of the human tongue. The profile is formed by ribs. The body also has a hose connection that communicates with the profile via multiple channels, so that loosened contaminations are suctioned away by negative pressure or vacuum via a drain hose connected to the hose connection.

It has been shown that not only in intensive care patients who are intubated in intensive care due to the severity of their illness and usually require ventilation, but also in normal patients or persons, the region of the mouth, nose and throat is spatially very limited and very sensitive to cleaning measures. In particular, the surface of the tongue can be injured during cleaning, so that there is a considerable risk of bleeding when cleaning the oral mucosa.

The problem with the known tongue cleaner is that the rigid profiles and the openings for suctioning the accumulated deposits are directed directly, i.e. perpendicularly, towards the tongue mucosa or perpendicularly to the plane or direction of movement of the tongue cleaner. As a result, injuries are possible due to the rigid profiles during cleaning and simultaneous suction. In particular, the openings directed vertically towards the tongue mucosa can create a negative pressure, causing the tongue cleaner to stick to the tongue mucosa, creating an additional risk of injury.

Therefore, the object of the present invention is to propose a tongue cleaner of the type described above, which enables atraumatic and at the same time powerful cleaning and suction.

### SUMMARY OF THE INVENTION

This object is achieved according to an exemplary embodiment of the invention by the features of claim 1. Further advantageous and claimed developments will be apparent from the dependent claims, the description, as well as the drawings.

Thus, a tongue cleaner for cleaning a tongue of a person or a patient, in particular a patient receiving intensive care, is proposed, comprising at least one main part and a handle section, wherein the main part is paired with at least one cleaning region for cleaning and suctioning deposits on the tongue. In order to achieve injury-safe and powerful cleaning and suction, it is provided that a first cleaning region has at least one elastic cleaning lip or the like, wherein the cleaning or scraping lip is paired with at least one suction opening such that the suction opening is aligned substantially at least parallel to the movement plane of the cleaning movement of the tongue cleaner.

In this way, a safe and powerful design of the tongue cleaner is realized to create a negative pressure for atraumatic suction. The cleaning lip, which is made of elastic material, safely and gently removes the secretion or tongue coating and completely and gently suctions it away through one or more suction openings, since the suction direction is aligned in the direction of movement of the tongue cleaner and thus approximately parallel to the surface of the tongue or the movement plane. The elastic, flexible cleaning lip can safely prevents accidental injury. Furthermore, an unintentional sticking suction of the tongue cleaner onto the surface of the tongue can also be reliably prevented by the suction openings not being aligned perpendicular to the surface of the tongue but at least parallel to the movement plane of the tongue cleaner or to the surface of the tongue.

Within the scope of an advantageous further development, it can be provided that a central axis of at least one suction opening is aligned approximately parallel to the movement plane of the direction of movement of the tongue cleaner and/or that a central axis of at least one suction opening lies in the movement plane of the cleaning movement of the tongue cleaner. This means that the central axes or center lines of the suction openings or holes are aligned approximately parallel to the tongue surface, which in turn is aligned approximately parallel to the movement plane. If the central axis of at least one suction opening lies in the movement plane, this is a special case of parallelism.

A particularly structurally simple design of an exemplary embodiment of the proposed tongue cleaner can be realized in that the main part has an approximately elongated housing recess or the like for collecting the detached deposits, wherein the housing recess has at least one edge region section running with a predetermined radius. The presence of the housing recess running with a radius or a predetermined curvature creates a region in which the deposits gently detached from the elastic cleaning lip can be collected in order to then be removed through the associated suction openings. Other constructive forms are also possible to realize atraumatic suction and cleaning.

Preferably, the elastic cleaning lip can protrude approximately perpendicularly from a front side of the main part and extend approximately in the predetermined radius of the edge region section of the housing recess. In this way, during the cleaning movement of the tongue cleaner along the movement plane, the cleaning lip can be moved along the surface of the tongue and the detached deposits can be collected almost automatically within the housing recess.

It is particularly advantageous that the cleaning lip is aligned approximately vertically on the front of the main part and has a curved, approximately U-shaped course which is virtually parallel to the edge region section of the housing recess. As a result, the vertically aligned cleaning lip enables good cleaning performance to be achieved. Due to the preferably U-shaped course along the edge region of the housing recess, all detached deposits and secretions are collected by the cleaning lip and fed together into the housing recess so that suction can take place there with the help of a generated negative pressure or a vacuum.

Accordingly, it is particularly advantageous that internal suction openings or the like are provided on the front side of the edge region section of the housing recess for suctioning the deposits accumulated in the housing recess. The arrangement of the suction openings on the front ensures a suction direction parallel to the movement plane of the cleaning direction or to the scraping movement of the tongue cleaner in order to reliably prevent suction on the tongue. Furthermore, the suction openings arranged on the edge region section of the housing recess are directly paired with to the cleaning lip, which enables good suction.

In a further development, it can be provided that external suction openings or the like are provided on a front edge region of the main part. These additional external suction openings achieve an improved cleaning effect, since any remaining detached deposits can be subsequently removed through the suction opening. In addition, this additional suction opening reliably prevents sticking suction even at maximum suction power. These external suction openings are also aligned parallel to the movement plane of the tongue cleaner and are directly paired with the cleaning lip.

In order to be able to connect the suction openings to an external suction for generating a predetermined negative pressure in a structurally simple manner, in an exemplary embodiment of the proposed tongue cleaner, it is provided that the inner suction openings and the outer suction openings are connected to a suction channel or the like extending within the main part, wherein the suction openings are connected via the suction channel to a connection for an external suction for generating a negative pressure.

A particularly structurally simple solution for controlling the suction power in an exemplary embodiment of the proposed tongue cleaner is realized within the scope of a preferred embodiment of the invention in that the handle section has an opening that can be covered by a finger of the operator or a so-called fingertip, which is connected to the suction channel for controlling the suction power. Only by closing the opening or by tapping with a finger or something similar does a negative pressure build up in the suction channel or at the suction openings, wherein the negative pressure can be individually controlled or terminated by the operator through the position of the finger on the opening. As a result, a good and cost-effective control for atraumatic suction is realized using simple means.

In order to further improve the cleaning performance of an exemplary embodiment of the proposed tongue cleaner, it can be provided, for example, that a cleaning sponge or the like is provided as a second cleaning region on the back of the main part. With the soft, elastic sponge, the tongue can be cleaned in advance and any deposits can be removed. Preferably, a cleaning sponge or cleaning knobs or brushes made of a soft, elastic material can be used. Since the second cleaning region is located on the back of the main part, cleaning with the second cleaning region can be done by simply turning the proposed tongue cleaner.

### BRIEF DESCRIPTION OF THE DRAWINGS

The present invention is further explained below with reference to the drawings.

In the drawings:

FIG. 1 is a schematic plan view of a front side of an exemplary embodiment of a tongue cleaner according to the invention;

FIG. 2 is a schematic plan view of a back side of the tongue cleaner;

FIG. 3 is a sectional view along the section line A1-A1 according to FIG. 1; and

FIG. 4 is a front view of the tongue cleaner.

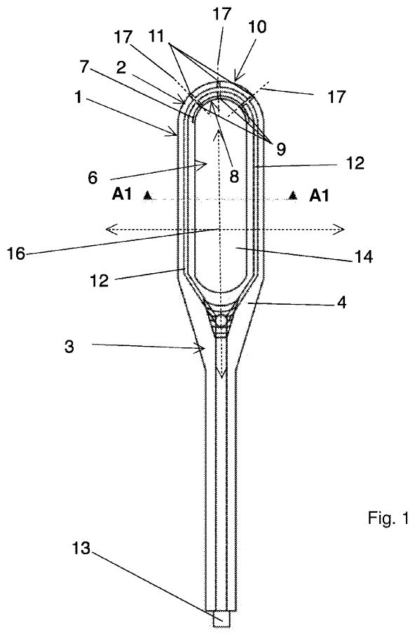

In FIGS. 1 to 4, various views of a tongue cleaner 1 according to the invention for cleaning the tongue of a person, in particular a patient receiving intensive care, are shown merely by way of example.

### DETAILED DESCRIPTION OF THE INVENTION

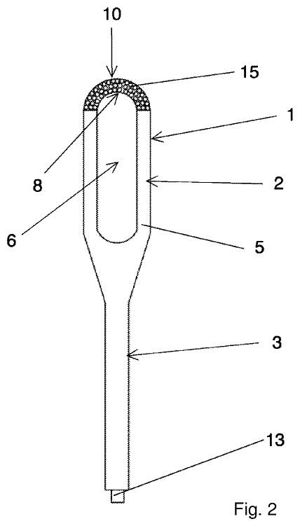

The tongue cleaner 1 of an exemplary embodiment of the present invention comprises a main part 2 and a handle section 3 for handling by an operator. The main part 2 has a front side 4 with a first cleaning region and a back side 5 with a second cleaning region. The main part 2 has an elongated housing recess 6 for collecting the detached deposits, which virtually forms a continuous opening in the main part 2. As can be seen in particular from FIGS. 1 and 2, the elongated housing recess 6 has an edge region section 8 on each of its front sides, which extends in a predetermined radius.

The first cleaning region provided on the front side of the main part 2 has an elastic cleaning lip 7. The cleaning lip 7 runs along the front side of the main part 2 along the curved edge region section 8 of the housing recess 6. The cleaning lip 7 is aligned approximately vertically on the front side of the main part 2 and has a curved, approximately U-shaped course parallel to the edge region section 8 of the housing recess 6.

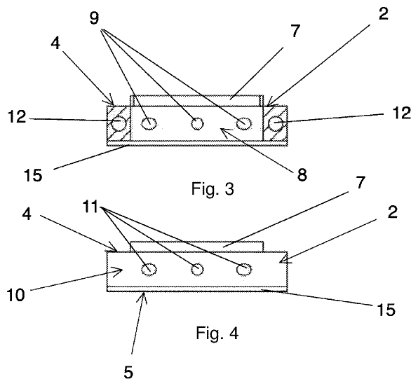

Accordingly, the edge region section 8 of the housing recess 6 forms an inner curvature, on which a plurality of inner suction openings 9 are arranged on the front side for suctioning the deposits accumulated within the housing recess 6. As can be seen in particular from FIG. 3, the first inner suction openings 9 are arranged symmetrically next to one another along the curved edge region section 8 of the housing recess.

External suction openings 11 are provided on a front edge region 10 of the main part 2 opposite the curved edge region section 8 of the housing recess 6, as shown in FIG. 4, for example. The front edge region 10 of the main part 2 forms practically an outer curvature of the main part 2 in the proposed tongue cleaner 1. The outer suction openings 11 are also arranged symmetrically next to one another along the front edge region 10.

According to FIG. 1, the inner suction openings 9 on the inner curvature or on the edge region section 8 of the housing recess 6 on the main part 2 and the outer suction openings 11 on the outer curvature or on the front edge region 10 of the main part 2 are directly paired with to the cleaning lip 7, since the cleaning lip 7 is arranged between the edge region section 8 of the housing recess 6 and the front edge region 10 on the main part 2.

Furthermore, in FIG. 1, the movement plane 16 of the direction of movement of the tongue cleaner 1 for cleaning is indicated by dashed double arrows arranged in a cross shape. The double arrows essentially span the movement plane 16 in the drawing plane. In order to suction the deposits detached from the tongue with the elastic cleaning lip 7, the inner suction openings 9 and the outer suction openings 11 are aligned approximately parallel to the movement plane 16 of the cleaning direction or scraping direction of the tongue cleaner 1. Accordingly, the central axes 17 of the suction openings 9, 11, indicated as dashed lines, are aligned parallel to the movement plane 16. Since the inner suction openings 9 and the outer suction openings 11 are located on the curved edge region section 8 or on the curved front edge region 10 of the main part 2, the center axes 17 of the suction openings 9, 11 are aligned at a predetermined angle to one another. Irrespective of this, the central axes 17 are either aligned parallel to the movement plane 16 or lie in the movement plane 16 of the tongue cleaner 1.

In order to suction the deposits, the inner suction openings 9 and the outer suction openings 11 are connected to a suction channel 12 which runs in the main part 2 of the tongue cleaner 1. The suction openings 9, 11 are connected via the suction channel 12 to a connection 13 for an external suction system (not shown). The suction channel 12 is indicated in FIG. 1 with a dashed line inside the main part 2 of the tongue cleaner 1.

In order to control the suction power, an opening 14 which can be covered by a finger of the operator is provided on the handle section 3 of the tongue cleaner 1 and is connected to the suction channel 12 for controlling the suction power. Preferably, the opening 14 on the front side of the main part 2 is provided like the first cleaning region with the cleaning lip 7 on the handle section 3, so that the operator can ergonomically easily operate or cover the opening 14 with his index finger.

As is particularly clear from FIG. 2, a second cleaning region with a cleaning sponge 15 is provided on the back of the main part 2 of the proposed tongue cleaner 1. Using the cleaning sponge 15 made of soft, elastic material, the operator can first loosen the deposits on the patient's tongue and then use the cleaning lip 7 to remove these deposits from the tongue through the suction openings 9, 11.

### REFERENCE NUMERALS

1 tongue cleaner
2 main part
3 handle section
4 front side of the main part
5 back side of the main part
6 housing recess
7 elastic cleaning lip
8 edge region section of the housing recess
9 internal suction opening
10 front edge region of the main part
11 external suction opening
12 suction channel
13 connection for external suction
14 opening for controlling the suction power
15 cleaning sponge
16 movement plane for cleaning
17 central axis of the suction opening

1.-10. (canceled)

11. A tongue cleaner for cleaning the tongue of a patient, comprising at least one main part and a handle section, wherein the main part is paired with at least one cleaning region for cleaning and suctioning deposits on the tongue, wherein a first cleaning region has at least one elastic cleaning lip, wherein the cleaning lip is paired with at least one suction opening such that the suction opening is aligned at least parallel to the movement plane of the cleaning movement.

12. The tongue cleaner according to claim 11, wherein a central axis of at least one suction opening is aligned approximately parallel to the movement plane of the cleaning movement of the tongue cleaner and/or wherein a central axis of at least one suction opening lies in the movement plane of the cleaning movement of the tongue cleaner.

13. The tongue cleaner according to claim 11, wherein the main part has an elongated housing recess for collecting the detached deposits, wherein the housing recess has at least one edge region section extending in a predetermined radius.

14. The tongue cleaner according to claim 13, wherein the elastic cleaning lip projects approximately perpendicularly from a front side of the main part and runs approximately in the predetermined radius of the edge region section of the housing recess.

15. The tongue cleaner according to claim 13, wherein inner suction openings are provided on the front side of the edge region section of the housing recess for suctioning the deposits accumulated in the housing recess.

16. The tongue cleaner according to claim 11, wherein external suction openings are provided on a front edge region of the main part.

17. The tongue cleaner according to claim 16, wherein the inner suction openings and the outer suction openings are connected to a suction channel extending within the main part, wherein the suction openings are connected via the suction channel to a connection for external suction.

18. The tongue cleaner according to claim 11, wherein the handle section has an opening that can be covered by a finger of the operator for controlling the suction power.

19. The tongue cleaner according to claim 18, wherein the coverable opening is connected to the suction channel.

20. The tongue cleaner according to claim 11, wherein a cleaning sponge is provided on a back side of the main part as a second cleaning region.

21. A method of tongue cleaning comprising turning on suction in a suction system;
connecting a tongue cleaner to the suction system;
creating negative pressure for atraumatic suction and cleaning;
applying tongue cleaner to a tongue;
collecting detached deposits; wherein the tongue cleaner comprises at least one main part and a handle section, wherein the main part is paired with at least one cleaning region for cleaning and suctioning deposits on the tongue, wherein a first cleaning region has at least one elastic cleaning lip, wherein the cleaning lip is paired with at least one suction opening such that the suction opening is aligned at least parallel to the movement plane of the cleaning movement.

22. The method of claim 21, wherein the tongue cleaner further comprises a central axis of at least one suction opening is aligned approximately parallel to the movement plane of the cleaning movement of the tongue cleaner and/or wherein a central axis of at least one suction opening lies in the movement plane of the cleaning movement of the tongue cleaner.

23. The method of claim 21, wherein the main part of the tongue cleaner has an elongated housing recess for collecting the detached deposits, wherein the housing recess has at least one edge region section extending in a predetermined radius.

24. The method of claim 23, wherein the elastic cleaning lip of the tongue cleaner projects approximately perpendicularly from a front side of the main part and runs approximately in the predetermined radius of the edge region section of the housing recess.

25. The method of claim 23, wherein inner suction openings of the tongue cleaner are provided on the front side of the edge region section of the housing recess for suctioning the deposits accumulated in the housing recess.

26. The method of claim 21, wherein external suction openings of the tongue cleaner are provided on a front edge region of the main part.

27. The method of claim 26, wherein the inner suction openings and the outer suction openings of the tongue cleaner are connected to a suction channel extending within the main part, wherein the suction openings are connected via the suction channel to a connection to the suction system.

28. The method of claim 21, wherein the handle section of the tongue cleaner has an opening that can be covered by a finger of the operator for controlling the suction power.

29. The method of claim 21, wherein a cleaning sponge is provided on a back side of the main part as a second cleaning region.

30. A tongue cleaning system comprising
a suction system,
a tongue cleaner connected to the suction system through a connector, wherein the tongue cleaner comprises at least one main part and a handle section, wherein the main part is paired with at least one cleaning region for cleaning and suctioning deposits on the tongue, wherein a first cleaning region has at least one elastic cleaning lip, wherein the cleaning lip is paired with at least one suction opening such that the suction opening is aligned at least parallel to the movement plane of the cleaning movement.
